(19) United States
(12) Patent Application Publication
(10) Pub. No.: US 2009/0264452 A1
(43) Pub. Date: Oct. 22, 2009

(54) 2-(Phenylamino)-Pyrimidin-5-Amides As Cannabinoid 2 Receptors Modulators for the Treatment of Immune or Inflammatory Disorders

(76) Inventors: Andrew John Eatherton, Essex (GB); Gerard Martin Paul Giblin, Essex (GB); William Leonard Mitchell, Essex (GB); Alan Naylor, Essex (GB); Derek Anthony Rawlings, Essex (GB); Ian David Wall, Essex (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 10/597,476

(22) PCT Filed: Feb. 1, 2005
(86) PCT No.: PCT/GB05/00341

§ 371 Date: Jul. 27, 2006

(30) Foreign Application Priority Data
Feb. 3, 2004 (GB) 0402356.0

(51) Int. Cl. A61K 31/505; C07D 239/42; C07D 405/12; A61K 31/506; A61P 29/00; A61P 37/00; A61P 19/00

(52) U.S. Cl. 514/275; 544/332

(57) ABSTRACT

The present invention relates to novel pyrimidine derivatives such as compounds of the formula (I):

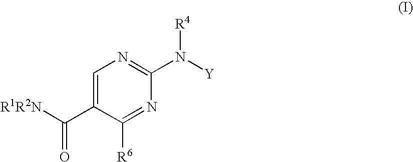

and the use of such compounds or pharmaceutical compositions thereof in the treatment of diseases, particularly pain, which are mediated by the activity of the cannabinoid 2 receptor.

The present invention relates to novel pyrimidine derivatives, pharmaceutical compositions containing these compounds and their use in the treatment of diseases, particularly pain, which diseases are caused directly or indirectly by an increase or decrease in activity of the cannabinoid receptor.

Cannabinoids are a specific class of psychoactive compounds present in Indian cannabis (Cannabis sativa), including about sixty different molecules, the most representative being cannabinol, cannabidiol and several isomers of tetrahydrocannabinol. Knowledge of the therapeutic activity of cannabis dates back to the ancient dynasties of China, where, 5,000 years ago, cannabis was used for the treatment of asthma, migraine and some gynaecological disorders. These uses later became so established that, around 1850, cannabis extracts were included in the US Pharmacopaeia and remained there until 1947.

Cannabinoids are known to cause different effects on various systems and/or organs, the most important being on the central nervous system and on the cardiovascular system. These effects include alterations in memory and cognition, euphoria, and sedation. Cannabinoids also increase heart rate and vary systemic arterial pressure. Peripheral effects related to bronchial constriction, immunomodulation, and inflammation have also been observed. The capability of cannabinoids to reduce intraocular pressure and to affect respiratory and endocrine systems is also well documented. See e.g. L. E. Hollister, Health Aspects of Cannabis, Pharmacological Reviews, Vol. 38, pp. 1-20, (1986). More recently, it was found that cannabinoids suppress the cellular and humoral immune responses and exhibit antiinflammatory properties. Wirth et al., Antiinflammatory Properties of Cannabichrome, Life Science, Vol. 26, pp. 1991-1995, (1980).

In spite of the foregoing benefits, the therapeutic use of cannabis is controversial, both due to its relevant psychoactive effects (causing dependence and addiction), and due to manifold side effects that have not yet been completely clarified. Although work in this field has been ongoing since the 1940's, evidence indicating that the peripheral effects of cannabinoids are directly mediated, and not secondary to a CNS effect, has been limited by the lack of receptor characterization, the lack of information concerning an endogenous cannabinoid ligand and, until recently, the lack of receptor subtype selective compounds.

The first cannabinoid receptor was found to be mainly located in the brain, in neural cell lines, and, only to a lesser extent, at the peripheral level. In view of its location, it was called the central receptor (“CB1”). See Matsuda et al., “Structure of a Cannabinoid Receptor and Functional Expression of the Cloned cDNA,”Nature, Vol. 346, pp. 561-564 (1990. The second cannabinoid receptor (“CB2”) was identified in the spleen, and was assumed to modulate the non psychoactive effects of the cannabinoids. See Munro et el., “Molecular Characterization of a Peripheral Receptor for Cannabinoids,” Nature. Vol. 365, pp. 61-65 (1993).

Recently, some compounds have been prepared which are capable of acting as agonists on both the cannabinoid receptors. For example, use of derivatives of dihydroxypyrrole-(1,2,3-d,e)-1,4-benzoxazine in the treatment of glaucoma and the use of derivatives of 1,5-diphenyl-pyrazole as immunomodulators or psychotropic agents in the treatment of various neuropathologies, migraine, epilepsy, glaucoma, etc are known. See U.S. Pat. No. 5,112,820 and EP 576357, respectively. However, because these compounds are active on both the CB1 and CB2 receptor, they can lead to serious psychoactive effects.

The foregoing indications and the preferential localization of the CB2 receptor in the immune system confirms a specific role of CB2 in modulating the immune and antiinflammatory response to stimuli of different sources.

The total size of the patient population suffering from pain is vast (almost 300 million), dominated by those suffering from back pain, osteo-arthritic pain and post-operative pain. Neuropathic pain (associated with neuronal lesions such as those induced by diabetes, HIV, herpes infection, or stroke) occurs with lower, but still substantial prevalence, as does cancer pain.

The pathogenic mechanisms that give rise to pain symptoms can be grouped into two main categories:
those that are components of inflammatory tissue responses (Inflammatory Pain);
those that result from a neuronal lesion of some form (Neuropathic Pain).

Chronic inflammatory pain consists predominantly of osteoarthritis, chronic low back pain and rheumatoid arthritis. The pain results from acute and on-going injury and/or inflammation. There may be both spontaneous and provoked pain.

There is an underlying pathological hypersensitivity as a result of physiological hyperexcitability and the release of inflammatory mediators which further potentiate this hyperexcitability. CB2 receptors are expressed on inflammatory cells (T cells, B cells, macrophages, mast cells) and mediate immune suppression through inhibition of cellular interaction/inflammatory mediator release. CB2 receptors may also be expressed on sensory nerve terminals and therefore directly inhibit hyperalgesia.

The role of CB2 in immunomodulation, inflammation, osteoporosis, cardiovascular, renal and other disease conditions is now being examined. In light of the fact that cannabinoids act on receptors capable of modulating different functional effects, and in view of the low homology between CB2 and CB1, the importance of developing a class of drugs selective for the specific receptor sub-type is evident. The natural or synthetic cannabinoids currently available do not fulfil this function because they are active on both receptors.

Based on the foregoing, there is a need for compounds which are capable of selectively modulating the receptor for cannabinoids and, therefore, the pathologies associated with such receptors. Thus, CB2 modulators offer a unique approach toward the pharmacotherapy of immune disorders, inflammation, osteoporosis, renal ischemia and other pathophysiological conditions.

The present invention provides novel pyrimidine derivatives of formula (I) and pharmaceutically acceptable derivatives thereof, pharmaceutical compositions containing these compounds or derivatives, and their use as CB2 receptor modulators, which are useful in the treatment of a variety of disorders.
The present invention further comprises a method for treating disease mediated by CB2 receptors in an animal, including humans, which comprises administering to an animal in need thereof an effective amount of a compound of formula (I) or a pharmaceutically acceptable derivative thereof.

The invention provides compounds of formula (I):

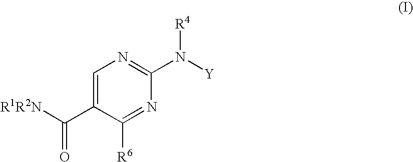

wherein:

Y is phenyl, unsubstituted or substituted with one, two or three substituents;

R¹ is selected from hydrogen, C₁₋₆ alkyl, C₃₋₆ cycloalkyl, or halosubstitutedC₁₋₆ alkyl;

R² is (CH₂)ₘR³ where m is 0 or 1;

or R¹ and R² together with N to which they are attached form an unsubstituted or substituted 4- to 8-membered non-aromatic heterocyclyl ring;

R³ is an unsubstituted or substituted 4- to 8-membered non-aromatic heterocyclyl group, an unsubstituted or substituted C₃₋₈ cycloalkyl group, an unsubstituted or substituted straight or branched C₁₋₁₀ alkyl, an unsubstituted or substituted C₅₋₇ cycloalkenyl or R⁵;

R⁴ is selected from hydrogen, C₁₋₆ alkyl, C₃₋₆ cycloalkyl, or halosubstitutedC₁₋₆ alkyl, COCH₃, or SO₂Me;

R⁵ is

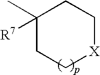

wherein p is 0, 1 or 2, and X is CH₂, O, S, SO or SO₂;

R⁶ is (C₃₋₆)cycloalkyl, 4- to 7-membered non aromatic heterocyclic group or unsubstituted C₂₋₆alkyl or substituted (C₁₋₆)alkyl;

R⁷ is OH, C₁₋₆alkoxy, NR^{8a}R^{8b}, NHCOR⁹, NHSO₂R⁹, SOqR⁹;

R^{8a} is H or C₁₋₆alkyl;

R^{8b} is H or C₁₋₆alkyl;

R⁹ is C₁₋₆alkyl;

q is 0, 1 or 2;

and pharmaceutically acceptable derivatives thereof,

wherein R⁶ is not CHxFn wherein n is 1, 2, or 3, x is 0, 1 or 2 and n and x add up to 3.

Compounds wherein R⁶ is CHxFn wherein n is 1, 2, or 3, x is 0, 1 or 2 and n and x add up to 3 are disclosed in copending International Application filed PCT/EP03/09217, published as WO 04/018433

In one embodiment X is CH₂, O or S.

In one embodiment Y is a substituted phenyl. In one embodiment Y is substituted by 1 or 2 substituents.

In one embodiment R¹ is hydrogen.

In one embodiment R⁴ is C₁₋₆ alkyl or hydrogen, in a further embodiment methyl or hydrogen, even more preferably hydrogen.

In one embodiment R⁶ is unsubstituted or substituted C₂₋₆alkyl, more preferably ethyl, isopropyl or tert butyl.

In one embodiment R⁷ is OH.

In one embodiment X is CH₂.

In one embodiment when R³ is an optionally substituted C₃₋₈cycloalkyl group or an optionally substituted 4- to 8-membered nonaromatic heterocyclyl group, m is 1.

In one embodiment R² is CH₂R₃.

In one embodiment R³ is an unsubstituted or substituted 4- to 8-membered non-aromatic heterocyclyl group.

When Y is substituted, the substituent or substituents may be selected from: C₁₋₆ alkyl, halosubstitutedC₁₋₆alkyl, C₁₋₆alkoxy, a hydroxy group, a cyano group, halo, a C₁₋₆alkyl sulfonyl group, —CONH₂, —NHCOCH₃, —COOH, halosubstituted C₁₋₆alkoxy, or SO₂NR^{8a}R^{8b} wherein R^{8a} and R^{8b} are as defined above.

In one embodiment Y is substituted by halo, cyano, methyl, trifluoromethyl, methoxy or trifluoromethoxy.

In one embodiment compounds of formula (I) are compounds of formula (Ia):

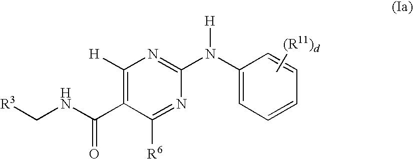

R³ is an unsubstituted or substituted 4- to 8-membered non-aromatic heterocyclyl group;

R⁶ is unsubstituted or substituted C₂₋₆alkyl;

R¹¹ is selected from halo, cyano, methyl, trifluoromethyl, methoxy or trifluoromethoxy;

d is 0, 1, 2 or 3;

and pharmaceutically acceptable derivatives thereof.

In one embodiment R³ is cyclohexyl or tetrahydropyran group.

In one embodiment R⁶ is ethyl, isopropyl or tert-butyl

When R¹ and R² together with N to which they are attached form a 4- to 8-membered non-aromatic heterocyclyl ring which is substituted, or when R³ is substituted, the substituent or substituents are preferably selected from: C₁₋₆ alkyl, C₁₋₆ alkoxy, a hydroxy group, a cyano group, halo or a sulfonyl group, methylsulfonyl, NR^{8a}R^{8b}, NHCOCH₃, (═O), CONHCH₃ and NHSO₂CH₃ wherein R^{8a} and R^{8b} are as described above.

When R¹ and R² together with N to which they are attached form a 4- to 8-membered non-aromatic heterocyclyl ring which is substituted, or when R³ is substituted there can be 1, 2 or 3 substituents.

When R⁶ is substituted by 1, 2 or 3 substitutents the substituent or substituents are preferably selected from OH, halo, cyano, C₁₋₆alkoxy, NR^{8a}R^{8b}, NHCOR⁹, NHSO₂R⁹, SOqR⁹; wherein R^{8a}, R^{8b}, R⁹, and q are defined above.

Preferably the compounds are 100 fold selective i.e. compounds of formula (I) have an EC50 value at the cloned human cannabinoid CB2 receptor of at least 100 times the EC50 values at the cloned human cannabinoid CB1 receptor or have less than 10% efficacy at the CB1 receptor.

The invention is described using the following definitions unless otherwise indicated.

The term “pharmaceutically acceptable derivative” means any pharmaceutically acceptable salt, ester, salt of such ester or solvate of the compounds of formula (I), or any other compound which upon administration to the recipient is capable of providing (directly or indirectly) a compound of formula (I) or an active metabolite or residue thereof.

It will be appreciated by those skilled in the art that compounds of formula (I) may be modified to provide pharmaceutically acceptable derivatives thereof at any of the functional groups in the compounds, and that the compounds of formula (I) may be derivatised at more than one position.

It will be appreciated that, for pharmaceutical use, the salts referred to above will be physiologically acceptable salts, but other salts may find use, for example in the preparation of compounds of formula (I) and the physiological acceptable salts thereof. Pharmaceutically acceptable salts include those described by Berge, Bighley and Monkhouse, J. Pharm. Sci., 1977, 66, 1-19. The term “pharmaceutically acceptable salts” includes salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, trishydroxylmethyl amino methane, tripropyl amine, tromethamine, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like.

Preferred examples of pharmaceutically acceptable salts include the ammonium, calcium, magnesium, potassium, and sodium salts, and those formed from maleic, fumaric, benzoic, ascorbic, pamoic, succinic, hydrochloric, sulfuric, bismethylenesalicylic, methanesulfonic, ethanedisulfonic, propionic, tartaric, salicylic, citric, gluconic, aspartic, stearic, palmitic, itaconic, glycolic, p-aminobenzoic, glutamic, benzenesulfonic, cyclohexylsulfamic, phosphoric and nitric acids.

The terms ‘halogen or halo’ are used to represent fluorine, chlorine, bromine or iodine.

The term ‘alkyl’ as a group or part of a group means a straight or branched chain alkyl group or combinations thereof, for example a methyl, ethyl, n-propyl, i-propyl, n-butyl, s-butyl, t-butyl, pentyl, hexyl, 1,1-dimethylethyl, or combinations thereof.

The term ‘alkoxy’ as a group or as part of a group means a straight, branched or cyclic chain alkyl group having an oxygen atom attached to the chain, for example a methoxy, ethoxy, n-propoxy, i-propoxy, n-butoxy, s-butoxy, t-butoxy group, pentoxy, hexyloxy group, cyclopentoxy or cyclohexyloxy group.

The term ‘cycloalkyl’ means a closed 4- to 8-membered non-aromatic ring, for example cyclobutyl, cyclopentyl, cyclohexyl or cycloheptyl, or cyclooctyl.

When R¹ and R² taken together with the N to which they are attached form an optionally substituted non-aromatic heterocyclyl ring, the ring may optionally contain 1, 2, 3 or 4 further hetero atoms. The ring may be saturated or unsaturated. Preferably the further hetero atoms are selected from oxygen, nitrogen or sulphur. An example of a 4 membered heterocyclyl ring is azetidinyl Examples of 5-membered heterocyclyl rings include pyrrolidinyl, Examples of 6-membered heterocyclyl rings are morpholinyl, piperizinyl or piperidinyl. An additional example is tetrahydropyridinyl. Examples of a 7-membered heterocyclyl ring are azapine or oxapine. Examples of 8-membered heterocyclyl rings are azacyclooctanyl, azaoxacyclooctanyl or azathiacyclooctanyl.

When R³ or R⁶ is an optionally substituted non-aromatic heterocyclyl group, the ring may contain 1, 2, 3, or 4 hetero atoms. Preferably the hetero atoms are selected from oxygen, nitrogen or sulphur. Examples of 4-membered groups are 2- or 3-azetidinyl, oxetanyl, thioxetanyl, thioxetanyl-s-oxide and thioxetanyl-s,s-dioxide. Examples of 5-membered heterocyclyl groups in this instance include dioxalanyl, pyrrolidinyl, tetrahydrofuranyl, tetrahydrothiophenyl and tetrahydrothiophenyl-s,s-dioxide. An additional example is tetrahydrothiophenyl-s-oxide. Examples of 6-membered heterocyclyl groups are morpholinyl, piperidinyl, piperazinyl, tetrahydropyranyl, tetrahydrothiopyranyl, thiomorpholinyl, thiomorpholinyl-s,s-dioxide, tetrahydropyridinyl, dioxanyl and tetrahydro-thiopyran-1,1-dioxide. Additional examples are tetrahydrothiopyranyl-s-oxide, tetrahydrothiopyranyl-s,s-dioxide, thiomorpholinyl-s-oxide and tetrahydro-thiopyran-1-oxide. Examples of 7-membered heterocyclyl rings are azapine and oxapine. Examples of 8-membered groups are azacyclooctanyl, azaoxacyclooctanyl, azathiacyclooctanyl, oxacylcooctanyl and thiacyclooctanyl. Additional examples are azathiacyclooctanyl-s-oxide, azathiacyclooctanyl-s,s-dioxide, thiacyclooctanyl-s-oxide and thiacyclooctanyl-s,s-dioxide

Preferred compounds of the present invention can be selected from:
2-(3-Chlorophenylamino)-4-isopropylpyrimidin-5-carboxylic acid cyclohexyl-methyl-amide;
2-(3-Chlorophenylamino)-4-isopropylpyrimidin-5-carboxylic acid (tetrahydro-pyran-4-ylmethyl)-amide;
2-(3-Chlorophenylamino)-4-ethylpyrimidin-5-carboxylic acid cyclohexyl-methyl-amide;
2-(3-Chlorophenylamino)-4-ethylpyrimidin-5-carboxylic acid (tetrahydro-pyran-4-ylmethyl)-amide,
   and pharmaceutically acceptable derivatives thereof.

Compounds of formula (I) can be prepared as set forth in the following scheme:

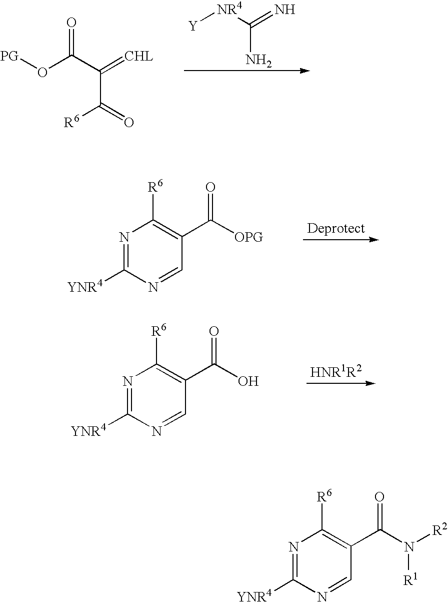

wherein L is a leaving group, for example halo, O—C₁₋₆alkyl, e.g. methoxy, ethoxy, or NR^{a}R^{b} wherein R^{a} or R^{b} are independently selected from C₁₋₆alkyl; PG is a protecting group for example methyl, ethyl or benzyl, and R¹, R², R⁴, R⁶, and Y are as defined for compounds of formula (I).

It is to be understood that the present invention encompasses all isomers of compounds of formula (I) and their pharmaceutically acceptable derivatives, including all geometric, tautomeric and optical forms, and mixtures thereof (e.g. racemic mixtures). Where additional chiral centres are present in compounds of formula (I), the present invention includes within its scope all possible diastereoismers, including mixtures thereof. The different isomeric forms may be separated or resolved one from the other by conventional methods, or any given isomer may be obtained by conventional synthetic methods or by stereospecific or asymmetric syntheses.

The subject invention also includes isotopically-labeled compounds, which are identical to those recited in formulas I and following, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ³H, ¹¹C, ¹⁴C, ¹⁸F, ¹²³I and ¹²⁵I.

Compounds of the present invention and pharmaceutically acceptable salts of said compounds that contain the aforementioned isotopes and/or other isotopes of other atoms are within the scope of the present invention. Isotopically-labeled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C; isotopes are particularly preferred for their ease of preparation and detectability. ¹¹C and ⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formula I and following of this invention can generally be prepared by carrying out the procedures disclosed in the Schemes and/or in the Examples below, by substituting a readily available isotopically labeled-reagent for a non-isotopically labeled reagent.

The compounds of formula (I) may be prepared in crystalline or non-crystalline form, and, if crystalline, may optionally be hydrated or solvated. This invention includes within its scope stoichiometric hydrates or solvates as well as compounds containing variable amounts of water and/or solvent.

The compounds of the invention bind selectively to the CB2 receptor, and may therefore useful in treating CB2 receptor mediated diseases.

In view of their ability to bind to the CB2 receptor, the compounds of the invention may be useful in the treatment of the disorders that follow. Thus, the compounds of formula (I) may be useful as analgesics. For example they may be useful in the treatment of chronic inflammatory pain (e.g. pain associated with rheumatoid arthritis, osteoarthritis, rheumatoid spondylitis, gouty arthritis and juvenile arthritis) including the property of disease modification and joint structure preservation; musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome; pain associated with myocardial ischemia; post operative pain; headache; toothache; and dysmenorrhea.

The compounds of the invention may also be useful disease modification or joint structure preservation in multiple sclerosis, rheumatoid arthritis, osteo-arthritis, rheumatoid spondylitis, gouty arthritis and juvenile arthritis.

The compounds of the invention may be particularly useful in the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. Neuropathic pain syndromes include: diabetic neuropathy; sciatica; non-specific lower back pain; multiple sclerosis pain; fibromyalgia; HIV-related neuropathy; post-herpetic neuralgia; trigeminal neuralgia; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are incredibly heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

The compounds of formula (I) may also be useful in the treatment of fever.

The compounds of formula (I) may also be useful in the treatment of inflammation, for example in the treatment of skin conditions (e.g. sunburn, burns, eczema, dermatitis, psoriasis); ophthalmic diseases such as glaucoma, retinitis, retinopathies, uveitis and of acute injury to the eye tissue (e.g. conjunctivitis); lung disorders (e.g. asthma, bronchitis, emphysema, allergic rhinitis, respiratory distress syndrome, pigeon fancier's disease, farmer's lung, chronic obstructive pulmonary disease, (COPD); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastroesophageal reflux disease); organ transplantation; other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, polymyositis, gingivitis, myocardial ischemia, pyrexia, systemic lupus erythematosus, tendinitis, bursitis, and Sjogren's syndrome.

The compounds of formula (I) may also be useful in the treatment of bladder hyperrelexia following bladder inflammation.

The compounds of formula (I) may also be useful in the treatment of immunological diseases such as autoimmune diseases, immunological deficiency diseases or organ transplantation. The compounds of formula (I) may also be effective in increasing the latency of HIV infection.

The compounds of formula (I) may also be useful in the treatment of diseases of abnormal platelet function (e.g. occlusive vascular diseases).

The compounds of formula (I) may also be useful in the treatment of neuritis, heart burn, dysphagia, pelvic hypersensitivity, urinary incontinence, cystitis or pruritis.

The compounds of formula (I) may also be useful for the preparation of a drug with diuretic action.

The compounds of formula (I) may also be useful in the treatment of impotence or erectile dysfunction.

The compounds of formula (I) may also be useful for attenuating the hemodynamic side effects of non-steroidal anti-inflammatory drugs (NSAID's) and cyclooxygenase-2 (COX-2) inhibitors.

The compounds of formula (I) may also be useful in the treatment of neurodegenerative diseases and neurodegeneration such as dementia, particularly degenerative dementia (including senile dementia, Alzheimer's disease, Pick's disease, Huntingdon's chorea, Parkinson's disease and Creutzfeldt-Jakob disease, motor neuron disease); vascular dementia (including multi-infarct dementia); as well as dementia associated with intracranial space occupying lesions; trauma; infections and related conditions (including HIV infection); dementia in Parkinson's disease; metabolism; toxins; anoxia and vitamin deficiency; and mild cognitive impairment associated with ageing, particularly Age Associated Memory Impairment. The compounds may also be useful for the treatment of amyotrophic lateral sclerosis (ALS) and neuroinflamation.

The compounds of formula (I) may also be useful in neuroprotection and in the treatment of neurodegeneration following stroke, cardiac arrest, pulmonary bypass, traumatic brain injury, spinal cord injury or the like.

The compounds of formula (I) may also be useful in the treatment of tinnitus.

The compounds of formula (I) may also be useful in the treatment of psychiatric disease for example schizophrenia, depression (which term is used herein to include bipolar depression, unipolar depression, single or recurrent major depressive episodes with or without psychotic features, catatonic features, melancholic features, atypical features or postpartum onset, seasonal affective disorder, dysthymic disorders with early or late onset and with or without atypical features, neurotic depression and social phobia, depression accompanying dementia for example of the Alzheimer's type, schizoaffective disorder or the depressed type, and depressive disorders resulting from general medical conditions including, but not limited to, myocardial infarction, diabetes, miscarriage or abortion, etc), anxiety disorders (including generalised anxiety disorder and social anxiety disorder), panic disorder, agoraphobia, social phobia, obsessive compulsive disorder and post-traumatic stress disorder, memory disorders, including dementia, amnesic disorders and age-associated memory impairment, disorders of eating behaviours, including anorexia nervosa and bulimia nervosa, sexual dysfunction, sleep disorders (including disturbances of circadian rhythm, dyssomnia, insomnia, sleep apnea and narcolepsy), withdrawal from abuse of drugs such as of cocaine, ethanol, nicotine, benzodiazepines, alcohol, caffeine, phencyclidine (phencyclidine-like compounds), opiates (e.g. cannabis, heroin, morphine), amphetamine or amphetamine-related drugs (e.g. dextroamphetamine, methylamphetamine) or a combination thereof.

The compounds of formula (I) may also be useful in preventing or reducing dependence on, or preventing or reducing tolerance or reverse tolerance to, a dependence-inducing agent. Examples of dependence inducing agents include opioids (e.g. morphine), CNS depressants (e.g. ethanol), psychostimulants (e.g. cocaine) and nicotine.

The compounds of formula (I) may also be useful in the treatment of kidney dysfunction (nephritis, particularly mesangial proliferative glomerulonephritis, nephritic syndrome), liver dysfunction (hepatitis, cirrhosis), gastrointestinal dysfunction (diarrhoea) and colon cancer.

The term “treatment” or “treating” as used herein includes the treatment of established disorders and also includes the prophylaxis thereof. The term “prophylaxis” is used herein to mean preventing symptoms in an already afflicted subject or preventing recurrence of symptoms in an afflicted subject and is not limited to complete prevention of an affliction.

According to a further aspect of the invention, we provide a compound of formula (I) or a pharmaceutically acceptable derivative thereof for use in human or veterinary medicine.

According to another aspect of the invention, we provide a compound of formula (I) or a pharmaceutically acceptable derivative thereof for use in the treatment of a condition which is mediated by the activity of cannabinoid 2 receptors.

According to a further aspect of the invention, we provide a method of treating a human or animal subject suffering from a condition which is mediated by the activity of cannabinoid 2 receptors which comprises administering to said subject a therapeutically effective amount of a compound of formula (I) or a pharmaceutically acceptable derivative thereof.

According to a further aspect of the invention we provide a method of treating a human or animal subject suffering from an immune disorder, an inflammatory disorder, pain, rheumatoid arthritis, multiple sclerosis, osteoarthritis or osteoporosis which method comprises administering to said subject an effective amount of a compound of formula (I) or a pharmaceutically acceptable derivative thereof.

Preferably the pain is selected from inflammatory pain, visceral pain, cancer pain, neuropathic pain, lower back pain, muscular sceletal, post operative pain, acute pain and migraine. More preferably the inflammatory pain is pain associated with rheumatoid arthritis or osteoarthritis.

According to another aspect of the invention is provided the use of a compound of formula (I) or a pharmaceutically acceptable derivative thereof for the manufacture of a therapeutic agent for the treatment or prevention of a condition such as an immune disorder, an inflammatory disorder, pain, rheumatoid arthritis, multiple sclerosis, osteoarthritis or osteoporosis.

In order to use a compound of formula (I) or a pharmaceutically acceptable derivative thereof for the treatment of humans and other mammals it is normally formulated in accordance with standard pharmaceutical practice as a pharmaceutical composition. Therefore in another aspect of the invention is provided a pharmaceutical composition comprising a compound of formula (I) or a pharmaceutically acceptable derivative thereof adapted for use in human or veterinary medicine.

As used herein, “modulator” means both antagonist, partial or full agonist and inverse agonist. Preferably the present modulators are agonists.

Compounds of formula (I) and their pharmaceutically acceptable derivatives may be administered in a standard manner for the treatment of the indicated diseases, for example orally, parenterally, sub-lingually, dermally, intranasally, transdermally, rectally, via inhalation or via buccal administration.

Compositions of formula (I) and their pharmaceutically acceptable derivatives which are active when given orally can be formulated as syrups, tablets, capsules and lozenges. A syrup formulation will generally consist of a suspension or solution of the compound or salt in a liquid carrier for example, ethanol, peanut oil, olive oil, glycerine or water with a flavouring or colouring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils, and are incorporated in a soft gelatin capsule shell.

Typical parenteral compositions consist of a solution or suspension of a compound or derivative in a sterile aqueous or non-aqueous carrier optionally containing a parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a compound of formula (I) or a pharmaceutically acceptable derivative thereof which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

Preferably the composition is in unit dosage form, for example a tablet, capsule or metered aerosol dose, so that the patient may administer a single dose.

Each dosage unit for oral administration contains suitably from 0.01 mg to 500 mg/Kg, and preferably from 0.01 mg to 100 mg/Kg, and each dosage unit for parenteral administration contains suitably from 0.001 mg to 100 mg/Kg, of a compound of formula (I) or a pharmaceutically acceptable derivative thereof calculated as the free acid. Each dosage unit for intranasal administration contains suitably 1-400 mg and preferably 10 to 200 mg per person. A topical formulation contains suitably 0.01 to 5.0% of a compound of formula (I).

The daily dosage regimen for oral administration is suitably about 0.01 mg/Kg to 1000 mg/Kg, of a compound of formula (I) or a pharmaceutically acceptable derivative thereof calculated as the free acid. The daily dosage regimen for parenteral administration is suitably about 0.001 mg/Kg to 200 mg/Kg, of a compound of formula (I) or a pharmaceutically acceptable derivative thereof calculated as the free acid. The daily dosage regimen for intranasal administration and oral inhalation is suitably about 10 to about 500 mg/person. The active ingredient may be administered from 1 to 6 times a day, sufficient to exhibit the desired activity.

It may be advantageous to prepare the compounds of the present invention as nanoparticles. This may improve the oral bioavailability of the compounds. For the purposes of the present invention “nanoparticulate” is defined as solid particles with 50% of the particles having a particle size of less than 1 μm, more preferably less than 0.75 μm

The particle size of the solid particles of compound (I) may be determined by laser diffraction. A suitable machine for determining particle size by laser diffraction is a Lecotrac laser particle size analyser, using an HELOS optical bench fitted with a QUIXEL dispersion unit.

Numerous processes for the synthesis of solid particles in nanoparticulate form are known. Typically these processes involve a milling process, preferably a wet milling process in the presence of a surface modifying agent that inhibits aggregation and/or crystal growth of the nanoparticles once created. Alternatively these processes may involve a precipitation process, preferably a process of precipitation in an aqueous medium from a solution of the drug in a non-aqueous solvent.

Accordingly, in a further aspect, the present invention provides a process for preparing compound (I) in nanoparticulate form as hereinbefore defined, which process comprises milling or precipitation.

Representative processes for the preparation of solid particles in nanoparticulate form are described in the patents and publications listed below.

U.S. Pat. No. 4,826,689 to Violanto & Fischer, U.S. Pat. No. 5,145,684 to Liversidge et al U.S. Pat. No. 5,298,262 to Na & Rajagopalan, U.S. Pat. No. 5,302,401 Liversidge et al U.S. Pat. No. 5,336,507 to Na & Rajagopalan, U.S. Pat. No. 5,340,564 to Illig & Sarpotdar U.S. Pat. No. 5,346,702 to Na Rajagopalan, U.S. Pat. No. 5,352,459 to Hollister et al U.S. Pat. No. 5,354,560 to Lovrecich, U.S. Pat. No. 5,384,124 to Courteille et al, U.S. Pat. No. 5,429,824 to June, U.S. Pat. No. 5,503,723 to Ruddy et al, U.S. Pat. No. 5,510,118 to Bosch et al, U.S. Pat. No. 5,518 to Bruno et al, U.S. Pat. No. 5,518,738 to Eickhoff et al, U.S. Pat. No. 5,534,270 to De Castro, U.S. Pat. No. 5,536,508 to Canal et al, U.S. Pat. No. 5,552,160 to Liversidge et al, U.S. Pat. No. 5,560,931 to Eickhoff et al, U.S. Pat. No. 5,560,932 to Bagchi et al, U.S. Pat. No. 5,565,188 to Wong et al, U.S. Pat. No. 5,571,536 to Eickhoff et al, U.S. Pat. No. 5,573,783 to Desieno & Stetsko, U.S. Pat. No. 5,580,579 to Ruddy et al, U.S. Pat. No. 5,585,108 to Ruddy et al, U.S. Pat. No. 5,587,143 to Wong, U.S. Pat. No. 5,591,456 to Franson et al, U.S. Pat. No. 5,622,938 to Wong, U.S. Pat. No. 5,662,883 to Bagchi et al, U.S. Pat. No. 5,665,331 to Bagchi et al, U.S. Pat. No. 5,718,919 to Ruddy et al, U.S. Pat. No. 5,747,001 to Wiedmann et al, WO93/25190, WO96/24336, WO97/14407, WO 98/35666, WO 99/65469, WO 00/18374, WO 00/27369, WO 00/30615 and WO 01/41760.

Such processes may be readily adapted for the preparation of compound (I) in nanoparticulate form. Such processes form a further aspect of the invention.

The process of the present invention preferably uses a wet milling step carried out in a mill such as a dispersion mill in order to produce a nanoparticulate form of the compound. The present invention may be put into practice using a conventional wet milling technique, such as that described in Lachman et al., The Theory and Practice of Industrial Pharmacy, Chapter 2, “Milling” p. 45 (1986).

In a further refinement, WO02/00196 (SmithKline Beecham plc) describes a wet milling procedure using a mill in which at least some of the surfaces are made of nylon (polyamide) comprising one or more internal lubricants, for use in the preparation of solid particles of a drug substance in nanoparticulate form.

In another aspect the present invention provides a process for preparing compounds of the invention in nanoparticulate form comprising wet milling a suspension of compound in a mill having at least one chamber and agitation means, said chamber(s) and/or said agitation means comprising a lubricated nylon, as described in WO02/00196.

The suspension of a compound of the invention for use in the wet milling is typically a liquid suspension of the coarse compound in a liquid medium. By “suspension” is meant that the compound is essentially insoluble in the liquid medium. Representative liquid media include an aqueous medium. Using the process of the present invention the average particle size of coarse compound of the invention may be up to 1 mm in diameter. This advantageously avoids the need to pre-process the compound.

In a further aspect of the invention the aqueous medium to be subjected to the milling comprises compound (I) present in from about 1% to about 40% w/w, preferably from about 10% to about 30% w/w, more preferably about 20% w/w.

The aqueous medium may further comprise one or more pharmaceutically acceptable water-soluble carriers which are suitable for steric stabilisation and the subsequent processing of compound (I) after milling to a pharmaceutical composition, e.g. by spray drying. Pharmaceutically acceptable excipients most suitable for steric stabilisation and spray-drying are surfactants such as poloxamers, sodium lauryl sulphate and polysorbates etc, stabilisers such as celluloses e.g. hydroxypropylmethyl cellulose; and carriers such as carbohydrates e.g. mannitol.

In a further aspect of the invention the aqueous medium to be subjected to the milling may further comprise hydroxypropylmethyl cellulose (HPMC) present from about 0.1 to about 10% w/w.

The process of the present invention may comprise the subsequent step of drying compound of the invention to yield a powder.

Accordingly, in a further aspect, the present invention provides a process for preparing a pharmaceutical composition contain a compound of the present invention which process comprises producing compound of formula (I) in nanoparticulate form optionally followed by drying to yield a powder.

A further aspect of the invention is a pharmaceutical composition comprising a compound of formula (I) or a pharmaceutically acceptable deriviate thereof in which the compound of formula (I) or a pharmaceutically acceptable deriviate thereof is present in solid particles in nanoparticulate form, in admixture with one or more pharmaceutically acceptable carriers or excipients.

By “drying” is meant the removal of any water or other liquid vehicle used during the process to keep compound of formula (I) in liquid suspension or solution. This drying step may be any process for drying known in the art, including freeze drying, spray granulation or spray drying. Of these methods spray drying is particularly preferred. All of these techniques are well known in the art. Spray drying/fluid bed granulation of milled compositions is carried out most suitably using a spray dryer such as a Mobile Minor Spray Dryer [Niro, Denmark], or a fluid bed drier, such as those manufactured by Glatt, Germany.

In a further aspect the invention provides a pharmaceutical composition as hereinbefore defined, in the form of a dried powder, obtainable by wet milling solid particles of compound of formula (I) followed by spray-drying the resultant suspension.

Preferably, the pharmaceutical composition as hereinbefore defined, further comprises HPMC present in less than 15% w/w, preferably in the range 0.1 to 10% w/w.

The CB₂ receptor compounds for use in the instant invention may be used in combination with other therapeutic agents, for example COX-2 inhibitors, such as celecoxib, deracoxib, rofecoxib, valdecoxib, parecoxib or COX-189; 5-lipoxygenase inhibitors; NSAID's, such as aspirin, diclofenac, indomethacin, nabumetone or ibuprofen; leukotriene receptor antagonists; DMARD's such as methotrexate; adenosine A1 receptor agonists; sodium channel blockers, such as lamotrigine; NMDA receptor modulators, such as glycine receptor antagonists; gabapentin and related compounds; tricyclic antidepressants such as amitriptyline; neurone stabilising antiepileptic drugs; mono-aminergic uptake inhibitors such as venlafaxine; opioid analgesics; local anaesthetics; 5HT₁ agonists, such as triptans, for example sumatriptan, naratriptan, zolmitriptan, eletriptan, frovatriptan, almotriptan or rizatriptan; EP₁ receptor ligands, EP₄ receptor ligands; EP₂ receptor ligands; EP₃ receptor ligands; EP₄ antagonists; EP₂ antagonists and EP₃ antagonists; bradykinin receptor ligands and vanilloid receptor ligand, antirheumatoid arthritis drugs, for example anti TNF drugs e.g. enbrel, remicade, anti-IL-1 drugs, or DMARDS e.g. leflunamide. When the compounds are used in combination with other therapeutic agents, the compounds may be administered either sequentially or simultaneously by any convenient route.

Additional COX-2 inhibitors are disclosed in U.S. Pat. No. 5,474,995 U. S. Pat. No. 5,633,272; U.S. Pat. No. 5,466,823, U.S. Pat. No. 6,310,099 and U.S. Pat. No. 6,291,523; and in WO 96/25405, WO 97/38986, WO 98/03484, WO 97/14691, WO99/12930, WO00/26216, WO00/52008, WO00/38311, WO01/58881 and WO02/18374.

The compound of the present invention may be administered in combination with other active substances such as 5HT3 antagonists, NK-1 antagonists, serotonin agonists, selective serotonin reuptake inhibitors (SSRI), noradrenaline re-uptake inhibitors (SNRI), tricyclic antidepressants and/or dopaminergic antidepressants.

Suitable 5HT3 antagonists which may be used in combination of the compound of the inventions include for example ondansetron, granisetron, metoclopramide.

Suitable serotonin agonists which may be used in combination with the compound of the invention include sumatriptan, rauwolscine, yohimbine, metoclopramide.

Suitable SSRIs which may be used in combination with the compound of the invention include fluoxetine, citalopram, femoxetine, fluvoxamine, paroxetine, indalpine, sertraline, zimeldine.

Suitable SNRIs which may be used in combination with the compound of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

Compounds of the present invention may used in combination with PDE4 inhibitors. The PDE4 inhibitor useful in this invention may be any compound that is known to inhibit the PDE4 enzyme or which is discovered to act in as PDE4 inhibitor, and which is only or essentially only a PDE4 inhibitor, not compounds which inhibit to a degree of exhibiting a therapeutic effect other members of the PDE family as well as PDE4. Generally it is preferred to use a PDE4 antagonists which has an IC₅₀ ratio of about 0.1 or greater as regards the IC₅₀ for the PDE4 catalytic form which binds rolipram with a high affinity divided by the IC₅₀ for the form which binds rolipram with a low affinity. Compounds of the present invention or combinations with PDE4 can be used in treating inflammation and as bronchodilators.

It turns out that there are at least two binding forms on human monocyte recombinant PDE 4 (hPDE 4) at which inhibitors bind. One explanation for these observations is that hPDE 4 exists in two distinct forms. One binds the likes of rolipram and denbufylline with a high affinity while the other binds these compound with a low affinity. The preferred PDE4 inhibitors of for use in this invention will be those compounds which have a salutary therapeutic ratio, i.e., compounds which preferentially inhibit cAMP catalytic activity where the enzyme is in the form that binds rolipram with a low affinity, thereby reducing the side effects which apparently are linked to inhibiting the form which binds rolipram with a high affinity. Another way to state this is that the preferred compounds will have an IC₅₀ ratio of about 0.1 or greater as regards the IC₅₀ for the PDE 4 catalytic form which binds rolipram with a high affinity divided by the IC₅₀ for the form which binds rolipram with a low affinity.

Reference is made to U.S. Pat. No. 5,998,428, which describes these methods in more detail. It is incorporated herein in full as though set forth herein.

Most preferred are those PDE4 inhibitors which have an IC₅₀ ratio of greater than 0.5, and particularly those compounds having a ratio of greater than 1.0.

A further aspect of the invention is an CB2 modulator in combination with a PDE4 inhibitor and pharmaceutical compositions comprising said combination.

A further aspect of the invention is a method of treating lung disorders for example asthma, bronchitis, emphysema, allergic rhinitis, respiratory distress syndrome, pigeon fancier's disease, farmer's lung, chronic obstructive pulmonary disease, (COPD) and cough or a disorder which can be treated with a broncodilator which comprises administering to a mammal including man, an effective amount of a CB modulator or a pharmaceutically acceptable derivative therefore and an effective amount of a PDE4 inhibitor or a pharmaceutically acceptable derivative thereof.

An additional aspect of the invention is the use of an effective amount of a CB2 modulator or a pharmaceutically acceptable derivative therefore and an effective amount of a PDE4 inhibitor or a pharmaceutically acceptable derivative thereof in the manufacture of a medicament in the treatment of lung disorders for example asthma, bronchitis, emphysema, allergic rhinitis, respiratory distress syndrome, pigeon fancier's disease, farmer's lung, chronic obstructive pulmonary disease, (COPD) and cough or for the manufacture of a brocodilator.

When used herein cough can have a number of forms and includes productive, non-productive, hyper-reactive, asthma and COPD associated.

A further aspect of the invention is a patient pack comprising an effective amount of a CB 2 modulator or a pharmaceutically acceptable derivative therefore and an effective amount of a PDE4 inhibitor or a pharmaceutically acceptable derivative

Preferred PDE4 compounds are cis [cyano-4-(3-cyclopentyloxy-4-methoxyphenyl)cyclohexan-1-carboxylate] also known as cilomilast or Ariflo®, 2-carbomethoxy-4-cyano-4-(3-cyclopropylmethoxy-4-difluoromethoxyphenyl)cyclohexan-1-one, and cis [4-cyano-4-(3-cyclopropylmethoxy-4-difluoromethoxyphenyl)cyclohexan-1-ol]. They can be made by the processed described in U.S. Pat. Nos. 5,449,686 and 5,552,438. Other PDE4 inhibitors, specific inhibitors, which can be used in this invention are AWD-12-281 from ASTA MEDICA (Hofgen, N. et al. 15th EFMC Int Symp Med Chem (September 6-10, Edinburgh) 1998, Abst P. 98); a 9-benzyladenine derivative nominated NCS-613 (INSERM); D-4418 from Chiroscience and Schering-Plough; a benzodiazepine PDE4 inhibitor identified as CI-1018 (PD-168787; Parke-Davis/Warner-Lambert); a benzodioxole derivative Kyowa Hakko disclosed in WO 9916766; V-11294A from Napp (Landells, L. J. et al. Eur Resp J [Annu Cong Eur Resp Soc (September 19-23, Geneva) 1998] 1998, 12(Suppl. 28): Abst P2393); roflumilast (CAS reference No 162401-32-3) and a pthalazinone (WO 99/47505) from Byk-Gulden (now Altana); or a compound identified as T-440 (Tanabe Seiyaku; Fuji, K. et al. J Pharmacol Exp Ther, 1998, 284(1): 162).

Additional PDE4 inhibitors are disclosed on pages 2 to 15 of WO01/13953. Specifically selected are arofylline, atizoram, BAY-19-8004, benafentrine, BYK-33043, CC-3052, CDP-840, cipamfylline, CP-220629, CP-293121, D-22888, D-4396, denbufylline, filaminast, GW-3600, ibudilast, KF-17625, KS-506-G, laprafylline, NA-0226A, NA-23063A, ORG-20241, ORG-30029, PDB-093, pentoxifylline, piclamilast, rolipram, RPR-117658, RPR-122818, RPR-132294, RPR-132703, RS-17597, RS-25344-000, SB-207499, SB210667, SB211572, SB-211600, SB212066, SB212179, SDZ-ISQ-844, SDZ-MNS-949, SKF-107806, SQ-20006, T-2585, tibenelast, tolafentrine, UCB-29646, V-11294A, YM-58997, YM-976 and zardaverine.

Preferably the PDE4 inhibitor is selected from cilomilast, AWD-12-281, NCS-613, D-4418, CI-1018, V-11294A, roflumilast or T-440.

It will be appreciated that the compounds of any of the above combinations or compositions may be administered simultaneously (either in the same or different pharmaceutical formulations), separately or sequentially.

The invention thus provides, in a further aspect, a combination comprising a compound of formula (I) or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

### Determination of Cannabinoid CB1 Receptor Agonist Activity

The cannabinoid CB1 receptor agonist activity of the compounds of formula (I) was determined in accordance with the following experimental method.

### Experimental Method

Yeast (Saccharomyces cerevisiae) cells expressing the human cannabinoid CB1 receptor were generated by integration of an expression cassette into the ura3 chromosomal locus of yeast strain MMY23. This cassette consisted of DNA sequence encoding the human CB1 receptor flanked by the yeast GPD promoter to the 5′ end of CB1 and a yeast transcriptional terminator sequence to the 3′ end of CB1. MMY23 expresses a yeast/mammalian chimeric G-protein alpha subunit in which the C-terminal 5 amino acids of Gpa1 are replaced with the C-terminal 5 amino acids of human Gαi3 (as described in Brown et al. (2000), Yeast 16:11-22). Cells were grown at 30° C. in liquid Synthetic Complete (SC) yeast media (Guthrie and Fink (1991), Methods in Enzymology, Vol. 194) lacking uracil, tryptophan, adenine and leucine to late logarithmic phase (approximately 6 OD₆₀₀/ml).

Agonists were prepared as 10 mM stocks in DMSO. EC₅₀ values (the concentration required to produce 50% maximal response) were estimated using dilutions of between 3- and 5-fold (BiomekFX, Beckman) into DMSO. Agonist solutions in DMSO (1% final assay volume) were transferred into black, clear bottom, microtitre plates from NUNC (96- or 384-well). Cells were suspended at a density of 0.2 OD₆₀₀/ml in SC media lacking histidine, uracil, tryptophan, adenine and leucine and supplemented with 10 mM 3-aminotriazole, 0.1M sodium phosphate pH 7.0, and 20 μM fluorescein di-β-D-glucopyranoside (FDGlu). This mixture (50 ul per well for 384-well plates, 200 ul per well for 96-well plates) was added to agonist in the assay plates (Multidrop 384, Labsystems). After incubation at 30° C. for 24 hours, fluorescence resulting from degradation of FDGlu to fluorescein due to exoglucanase, an endogenous yeast enzyme produced during agonist-stimulated cell growth, was determined using a Spectrofluor microtitre plate reader (Tecan; excitation wavelength: 485 nm; emission wavelength: 535 nm). Fluorescence was plotted against compound concentration and iteratively curve fitted using a four parameter fit to generate a concentration effect value. Efficacy (Eₘₐₓ) was calculated from the equation

Eₘₐₓ=Max_{[compound X]}−Min_{[compound X]}/Max_{[HU210]}−Min_{[HU210]}×100%

where Max_{[compound X]} and Min_{[Compound X]} are the fitted maximum and minimum respectively from the concentration effect curve for compound X, and Max_{[HU210]} and Min_{[HU210]} are the fitted maximum and minimum respectively from the concentration effect curve for (6aR,10aR)-3-(1,1′-Dimethylheptyl)-6a,7,10,10a-tetrahydro-1-hydroxy-6,6-dimethyl-6H-dibenzo[b,d]pyran-9-methanol (HU210; available from Tocris). Equieffective molar ratio (EMR) values were calculated from the equation

EMR=EC_{50 [compound X]}/EC_{50 [HU210]}

Where EC_{50 [compound X]} is the EC₅₀ of compound X and EC_{50 [HU210]} is the EC₅₀ of HU210.

Compounds of the Examples tested according to this method had EC₅₀ values>30,000 nM at the cloned human cannabinoid CB1 receptor.

### Determination of Cannabinoid CB2 Receptor Agonist Activity

The cannabinoid CB2 receptor agonist activity of the compounds of formula (I) was determined in accordance with the following experimental method.

### Experimental Method

Yeast (Saccharomyces cerevisiae) cells expressing the human cannabinoid CB2 receptor were generated by integration of an expression cassette into the ura3 chromosomal locus of yeast strain MMY23. This cassette consisted of DNA sequence encoding the human CB2 receptor flanked by the yeast GPD promoter to the 5′ end of CB2 and a yeast transcriptional terminator sequence to the 3′ end of CB2. MMY23 expresses a yeast/mammalian chimeric G-protein alpha subunit in which the C-terminal 5 amino acids of Gpa1 are replaced with the C-terminal 5 amino acids of human Gαi3 (as described in Brown et al. (2000), Yeast 16:11-22). Cells were grown at 30° C. in liquid Synthetic Complete (SC) yeast media (Guthrie and Fink (1991), Methods in Enzymology, Vol. 194) lacking uracil, tryptophan, adenine and leucine to late logarithmic phase (approximately 6 OD₆₀₀/ml).

Agonists were prepared as 10 mM stocks in DMSO. EC₅₀ values (the concentration required to produce 50% maximal response) were estimated using dilutions of between 3- and 5-fold (BiomekFX, Beckman) into DMSO. Agonist solutions in DMSO (1% final assay volume) were transferred into black, clear bottom, microtitre plates from NUNC (96- or 384-well). Cells were suspended at a density of 0.2 OD₆₀₀/ml in SC media lacking histidine, uracil, tryptophan, adenine and leucine and supplemented with 10 mM 3-aminotriazole, 0.1M sodium phosphate pH 7.0, and 20M fluorescein di-β-D-glucopyranoside (FDGlu). This mixture (50 ul per well for 384-well plates, 200 ul per well for 96-well plates) was added to agonist in the assay plates (Multidrop 384, Labsystems). After incubation at 30° C. for 24 hours, fluorescence resulting from degradation of FDGlu to fluorescein due to exoglucanase, an endogenous yeast enzyme produced during agonist-stimulated cell growth, was determined using a Spectrofluor microtitre plate reader (Tecan; excitation wavelength: 485 nm; emission wavelength: 535 nm). Fluorescence was plotted against compound concentration an iteratively curve fitted using a four parameter fit to generate a concentration effect value. Efficacy (Eₘₐₓ) was calculated from the equation

Eₘₐₓ=Max_{[compound X]}−Min_{[compound X]}/Max_{[HU210]}−Min_{[HU210]}×100%

where Max_{[compound X]} and Min_{[compound X]} are the fitted maximum and minimum respectively from the concentration effect curve for compound X, and Max_{[HU210]} and Min_{[HU210]} are the fitted maximum and minimum respectively from the concentration effect curve for (6aR,10aR)-3-(1,1′-Dimethylheptyl)-6a,7,10,10a-tetrahydro-1-hydroxy-6,6-dimethyl-6H-dibenzo[b,d]pyran-9-methanol (HU210; available from Tocris). Equieffective molar ratio (EMR) values were calculated from the equation

EMR=EC_{50 [compound X]}/EC_{50 [HU210]}

Where EC_{50 [compound X]} is the EC₅₀ of compound X and EC_{50 [HU210]} is the EC₅₀ of HU210.

The compounds of Example 1 to 3 tested according to this method had EC₅₀ values of <300 nm and efficacy value of >50% at the cloned human cannabinoid CB2 receptor.

The compound of Example 4 tested according to this method had an EC₅₀ value between 300 nM and 1000 nM and efficacy value of >50% at the cloned human cannabinoid CB2 receptor.

The compound of Example 5 tested according to this method had an EC₅₀ value of >1000 nM and efficacy value of <50% at the cloned human cannabinoid CB2 receptor.

The following examples are illustrative, but not limiting of the embodiments of the present invention.

### Conditions, Hardware, and Software Used for Mass-Directed Autopurification

### Hardware

Waters 600 gradient pump, Waters 2700 sample manager, Waters Reagent Manager, Micromass ZMD mass spectrometer, Gilson 202—fraction collector, Gilson Aspec—waste collector.

### Software

Micromass Masslynx version 3.5

### Column

The column used is typically a Supelco ABZ+ column whose dimensions are 10 mm internal diameter by 100 mm in length. The stationary phase particle size is 5 μm.

### Solvents

A. Aqueous solvent=Water+0.1% Formic Acid
B. Organic solvent=MeCN: Water 95:5+0.05% Formic Acid
Make up solvent=MeOH: Water 80:20+50 mMol Ammonium Acetate
Needle rinse solvent=MeOH: Water: DMSO 80:10:10

### Methods

Five methods are used depending on the analytical retention time of the compound of interest. They all have a flow rate of 20 ml/min and a 15-minute runtime, which comprises of a 10-minute gradient followed by a 5-minute column flush and re-equilibration step.

### Method 1 MDP 1.5-2.2=0-30% B

### Method 2 MDP 2.0-2.8=5-30% B

### Method 3 MDP 2.5-3.0=15-55% B

### Method 4 MDP 2.8-4.0=30-80% B

### Method 5 MDP 3.8-5.5=50-90% B

### Conditions Used for Analytical LCMS Systems

### Hardware

Agilent 1100 gradient pump

### Agilent 1100 Autosampler

### Agilent 1100 PDA Detector

### Agilent 1100 Degasser

Micromass ZQ mass spectrometer

### PL-ELS 1000

### Software

Micromass Masslynx versions 3.5/4.0

### Column

The column used is a Supelcosil ABZ+PLUS, the dimensions of which are 4.6 mm×33 mm. The stationary phase particle size is 3 m.

### Solvents

A: Aqueous solvent=10 mMol Ammonium Acetate+0.1% Formic Acid
B: Organic solvent=95% Acetonitrile+0.05% Formic Acid

### Method

The generic method used has 5.5 minute runtime, which comprises of a 4.7-minute gradient (0-100% B) followed by a 0.6 minute column flush and 0.2 minute re-equilibration step.

### Flow Rate

The above method has a flow rate of 3 ml/mins

### Conditions Used for NMR

### Hardware

### Bruker 400 MHz Ultrashield

### Bruker B-ACS60 Autosampler

### Bruker Advance 400 Console

### Software

User interface—NMR Kiosk
Controlling software—XWin NMR version 3.0

### Description 1: Ethyl 2-(3-chlorophenylamino)-4-isopropylpyrimidine-5-carboxylate

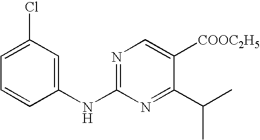

To a solution of 3-chlorophenylguanidine nitrate (0.58 g, Ref.: Ciba-Geigy Patent Application WO 95/09851, (1995)) in ethanol (10 ml) was added sodium ethoxide (0.18 g) and the mixture stirred for 3 minutes. Ethyl (N,N-dimethylamino)methylene isobutyrylacetate (0.54 g, Ref.: SmithKline Beecham Patent Application WO 01/32626, (2001)) was added and the mixture stirred under reflux for 2 hours. Ethanol was removed under reduced pressure and water (15 ml) added. The mixture was extracted with ethyl acetate (3×10 ml) and the combined extracts washed with brine (5 ml), dried (MgSO₄) and evaporated to afford, after trituration with isohexane, ethyl 2-(3-chlorophenylamino)-4-isopropylpyrimidine-5-carboxylate (0.35 g).

NMR (CDCl₃) δ 1.30 (6H, d), 1.40 (3H, t), 4.02 (1H, m), 4.36 (2H, q), 7.05 (1H, d), 7.26 (1H, t), 7.35 (1H, s), 7.42 (1H, d), 8.01 (1H, s), 8.91 (1H, s).

LC/MS, t=4.04 min, [MH⁺] 320.

### Description 2: 2-(3-Chlorophenylamino)-4-isopropylpyrimidine-5-carboxylic acid

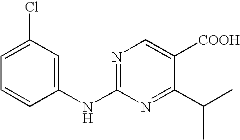

To a solution of ethyl 2-(3-chlorophenylamino)-4-isopropylpyrimidine-5-carboxylate (Description 1) (0.35 g) in ethanol (5 ml) was added a solution of potassium hydroxide (190 mg) in ethanol (4 ml) and the solution stirred at reflux for 4 h. Ethanol was removed under reduced pressure and water (15 ml) was added. The solution was washed with ether and concentrated hydrochloric acid was added to adjust the acidity to pH1. The precipitated solid was filtered, washed with water and dried in vacuo at 50° C. to afford 2-(3-chlorophenylamino)-4-isopropylpyrimidine-5-carboxylic acid (0.27 g).

NMR (DMSO-d6) δ 1.24 (6H, d), 4.06 (1H, m), 7.05 (1H, d), 7.34 (1H, t), 7.67 (1H, d), 8.14 (1H, s), 8.86 (1H, s), 10.25 (1H, s), 13.0 (1H, br s).

LC/MS, t=3.84 min, [MH⁺] 292

### Description 3: Ethyl 2-(3-chlorophenyl-amino)-4-ethylpyrimidine-5-carboxylate

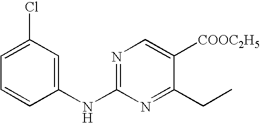

In a manner similar to Description 1, 3-chlorophenylguanidine nitrate (0.58 g) and ethyl(N,N-dimethylamino)methylene propionylacetate (0.50 g, Ref.: SmithKline Beecham Patent Application WO 01/32626, (2001)) afforded ethyl 2-(3-chlorophenyl-amino)-4-ethylpyrimidine-5-carboxylate (0.70 g).

NMR (CDCl₃) δ 1.33 (3H, t), 1.40 (3H, t), 3.16 (2H, q), 4.36 (2H, q), 7.06 (1H, d), 7.26 (1H, t), 7.36 (1H, s), 7.44 (1H, d), 7.96 (1H, s), 8.94 (1H, s).

LC/MS, t=3.93 min, [MH⁺]306.

### Description 4: 2-(3-Chlorophenylamino)-4-ethyl-pyrimidine-5-carboxylic acid

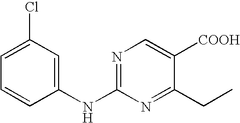

In a manner similar to Description 2, ethyl 2-(3-chlorophenylamino)-4-ethylpyrimidine-5-carboxylate (0.69 g) afforded 2-(3-chlorophenylamino)-4-ethyl-pyrimidine-5-carboxylic acid (0.56 g).

NMR (DMSO-d6) δ 1.25 (3H, t), 3.09 (2H, q), 7.05 (1H, d), 7.34 (1H, t), 7.71 (1H, d), 8.07 (1H, s), 8.88 (1H, s), 10.25 (1H, s), 12.95 (1H, br s).

LC/MS, t=3.70 min, [MH⁺]278.

### Description 5: Ethyl (2Z)-3-(dimethylamino)-2-[2-(dimethylamino)propanoyl]-2-propenoate

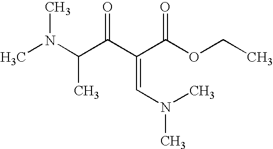

A mixture of ethyl 4-(dimethylamino)-3-oxopentanoate (1.54 g) (Hosmer et al, J Org Chem (1985), 50(19), 3627) and N,N-dimethylformamide dimethyl acetal (3 ml) was refluxed for 2.5 hrs. The cooled mixture was added to ethyl acetate (20 ml) and brine (20 ml) and the organic layer was washed with brine, dried (MgSO₄) and evaporated to give the title compound (1.17 g).

NMR (DMSO-d6) δ 1.11 (3H, m), 1.33 (3H, t), 2.21 (6H, m), 3.0 (6H, broad), 3.75 (1H, m), 4.15 (1H, m), 4.25 (1H, m), 7.69 (1H, d).

### Description 6: Ethyl 2-[(3-chlorophenyl)amino]-4-[1-(dimethylamino)ethyl]-5-pyrimidinecarboxylate

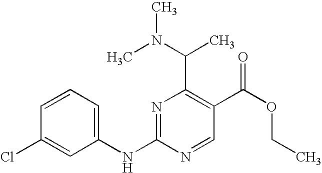

A mixture of ethyl (2Z)-3-(dimethylamino)-2-[2-(dimethylamino)propanoyl]-2-propenoate (606 mg), N-(3-chlorophenyl)guanidine nitrate (prepared as in WO 95/09851, 640 mg) and sodium ethoxide (187 mg) in ethanol (15 ml) was refluxed for 2 hrs. The solvent was evaporated and the residue dissolved in ethyl acetate (30 ml) and water (30 ml). The organic layer was washed with water, brine, dried (MgSO₄) and evaporated. Purification by chromatography on silica gel (dichloromethane/ether 30:1) gave the title compound (518 mg).

LC/MS t=2.03 min, Molecular ion observed (MH+)=349 consistent with the molecular formula C₁₇H₂₁³⁵ClN₄O₂

### Description 7: 2-[(3-Chlorophenyl)amino]-4-[1-(dimethylamino)ethyl]-5-pyrimidinecarboxylic acid hydrochloride

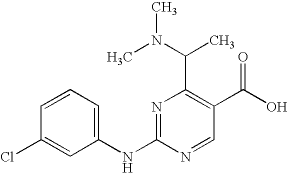

Ethyl 2-[(3-chlorophenyl)amino]-4-[1-(dimethylamino)ethyl]-5-pyrimidinecarboxylate (495 mg) was dissolved in ethanol (15 ml), a solution of potassium hydroxide (239 mg) in water (15 ml) added and the mixture refluxed for 3 hrs. The solution was evaporated to approx 4 ml, acidified with concentrated hydrochloric acid, then evaporated to dryness. The residue was taken up in ethanol (5 ml), filtered and the filtrate was evaporated to an oil which was triturated with ether to give the title compound (500 mg) as a solid.

LC/MS t=2.21 min, Molecular ion observed (MH+)=321 consistent with the molecular formula C₁₅H₁₇³⁵ClN₄O₂

### EXAMPLE 1

### 2-(3-Chlorophenylamino)-4-isopropylpyrimidin-5-carboxylic acid cyclohexyl-methyl-amide

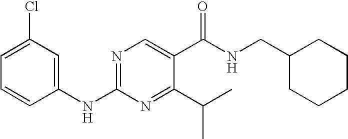

To a solution of 2-(3-chlorophenylamino)-4-isopropylpyrimidine-5-carboxylic acid (Description 2) (31.5 mg) in dimethylformamide (1.5 ml) was added successively N-ethylmorpholine (42 μl), cyclohexanemethylamine (14 mg), 1-hydroxybenzotriazole hydrate (25 mg) and 1-(3-dimethylamino-propyl)-3-ethylcarbodiimide hydrochloride (25 mg). The solution was stirred for 3 h and allowed to stand overnight. Dimethylformamide was removed under reduced pressure and ethyl acetate (5 ml) added. The solution was washed sequentially with 5% sodium bicarbonate solution (2.5 ml), water (2.5 ml), 5% citric acid solution (2.5 ml) and brine (2×2.5 ml), dried (MgSO₄) and evaporated to afford the title compound (32 mg).

NMR (DMSO-d6) δ 0.85-1.0 (2H, m), 1.1-1.3 (3H, m), 1.22 (6H, d), 1.51 (1H, m), 1.55-1.8 (5H, m), 3.07 (2H, t), 3.49 (1H, m), 7.00 (1H, d), 7.31 (1H, t), 7.65 (1H, d), 8.14 (1H, s), 8.41 (2H, m), 10.0 (1H, s).

LC/MS, t=4.03 min, [MH⁺] 387.

### EXAMPLE 2

### 2-(3-Chlorophenylamino)-4-isopropylpyrimidin-5-carboxylic acid (tetrahydro-pyran-4-ylmethyl)-amide

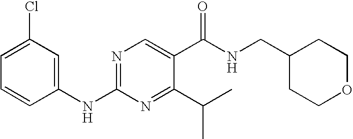

In a manner similar to Example 1,2-(3-chlorophenylamino)-4-isopropylpyrimidine-5-carboxylic acid (Description 2) (31.5 mg) and 4-aminomethyltetrahydropyran (14.5 mg) afforded the title compound (36 mg).

NMR (DMSO-d6) δ 1.15-1.3 (2H, m), 1.22 (6H, d), 1.62 (2H, d), 1.76 (1H, m), 3.13 (2H, t), 3.28 (2H, t), 3.49 (1H, m), 3.86 (2H, d of d), 7.00 (1H, d), 7.31 (1H, t), 7.65 (1H, d), 8.15 (1H, s), 8.44 (1H, s), 8.46 (1H, t), 10.0 (1H, s).

LC/MS t=3.43 min, [MH^{+]}389.

### EXAMPLE 3

### 2-(3-Chlorophenylamino)-4-ethylpyrimidin-5-carboxylic acid cyclohexyl-methyl-amide

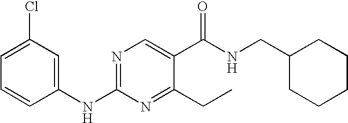

In a manner similar to Example 1, 2-(3-chlorophenylamino)-4-ethylpyrimidine-5-carboxylic acid (Description 4) (30 mg) and cyclohexanemethylamine (16 μl) afforded the title compound (28 mg). NMR (DMSO-d6) δ 0.85-1.0 (2H, m), 1.1-1.3 (3H, m), 1.22 (3H, t), 1.51 (1H, m), 1.55-1.8 (5H, m), 2.84 (2H, q), 3.07 (2H, t), 7.00 (1H, d), 7.31 (1H, t), 7.68 (1H, d), 8.08 (1H, s), 8.37 (1H, t), 8.46 (1H, s), 10.0 (1H, s).

LC/MS t=3.92 min, [MH⁺] 373.

### EXAMPLE 4

### 2-(3-Chlorophenylamino)-4-ethylpyrimidin-5-carboxylic acid (tetrahydro-pyran-4-ylmethyl)-amide

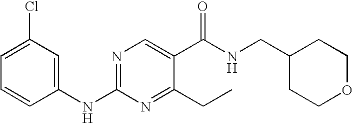

In a manner similar to Example 1, 2-(3-chlorophenylamino)-4-ethylpyrimidine-5-carboxylic acid (Description 4) (30 mg) and 4-aminomethyltetrahydropyran (14.5 mg) afforded the title compound (32 mg).

NMR (DMSO-d6) δ 1.1-1.25 (2H, m), 1.23 (3H, t), 1.62 (2H, d), 1.76 (1H, m), 2.85 (2H, q), 3.13 (2H, t), 3.28 (2H, t), 3.86 (2H, d), 7.01 (1H, d), 7.31 (1H, t), 7.68 (1H, d), 8.08 (1H, s), 8.43 (1H, s), 8.48 (1H, t), 10.0 (1H, s).

LC/MS t=3.29 min, [MH⁺]375.

### EXAMPLE 5

### 2-[(3-chlorophenyl)amino]-4-[1-(dimethylamino)ethyl]-N-(tetrahydro-2H-pyran-4-ylmethyl)-5-pyrimidinecarboxamide hydrochloride

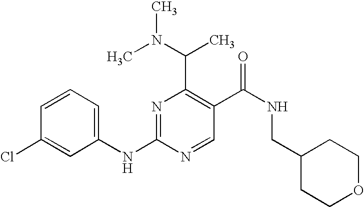

A solution of 2-[(3-chlorophenyl)amino]-4-[1-(dimethylamino)ethyl]-5-pyrimidinecarboxylic acid hydrochloride (57 mg), (tetrahydro-2H-pyran-4-ylmethyl)amine (18.5 mg), N,N-diisopropylethylamine (52 mg), 1-hydroxybenzotriazole hydrate (27 mg) and 1-(3-dimethylamino-propyl)-3-ethylcarbodiimide hydrochloride (38 mg) in dimethylformamide (1 ml) was allowed to stand at room temperature overnight. Dimethylformamide was removed under reduced pressure and ethyl acetate added. The solution was washed with sodium bicarbonate solution, water (×2), and evaporated to dryness. The residue was taken up in dichloromethane, treated with excess ethereal hydrogen chloride and evaporated to a gum which was triturated with ether to give the title compound (51 mg) as a solid.

LC/MS t=2.3 min, Molecular ion observed (MH+)=418 consistent with the molecular formula C₂₁H₂₈³⁵ClN₅O₂

Formulations for pharmaceutical use incorporating compounds of the present invention can be prepared in various forms and with numerous excipients. Examples of such formulations are given below.

### EXAMPLE 6

### Inhalant Formulation

A compound of formula (I) or a pharmaceutically acceptable derivative thereof, (1 mg to 100 mg) is aerosolized from a metered dose inhaler to deliver the desired amount of drug per use.

### EXAMPLE 7

### Tablet Formulation

| Tablets/Ingredients | | Per Tablet |
|---|---|---|
| 1. | Active ingredient (Compound of formula (I) | 40 mg |
| | or pharmaceutically acceptable derivative) | |
| 2. | Corn Starch | 20 mg |
| 3. | Alginic acid | 20 mg |
| 4. | Sodium Alginate | 20 mg |
| 5. | Mg stearate | 1.3 mg |

### Procedure for Tablet Formulation:

Ingredients 1, 2, 3 and 4 are blended in a suitable mixer/blender. Sufficient water is added portion-wise to the blend with careful mixing after each addition until the mass is of a consistency to permit its conversion to wet granules. The wet mass is converted to granules by passing it through an oscillating granulator using a No. 8 mesh (2.38 mm) screen. The wet granules are then dried in an oven at 140° F. (60° C.) until dry. The dry granules are lubricated with ingredient No. 5, and the lubricated granules are compressed on a suitable tablet press.

### EXAMPLE 8

### Parenteral Formulation

A pharmaceutical composition for parenteral administration is prepared by dissolving an appropriate amount of a compound of formula (I) in polyethylene glycol with heating. This solution is then diluted with water for injections Ph Eur. (to 100 ml). The solution is then rendered sterile by filtration through a 0.22 micron membrane filter and sealed in sterile containers.

What is claimed is:

1. A compound of formula (I); 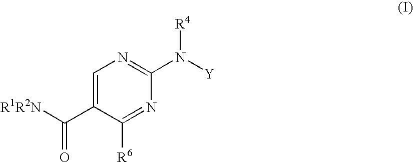
wherein:
Y is phenyl, unsubstituted or substituted with one, two or three substituents;
R¹ is selected from hydrogen, C₁₋₆ alkyl, C₃₋₆ cycloalkyl, or halosubstitutedC₁₋₆ alkyl;
R² is (CH₂)ₘR³ where m is 0 or 1;
or R¹ and R² together with N to which they are attached form an unsubstituted or substituted 4- to 8-membered nonaromatic heterocyclyl ring;
R³ is an unsubstituted or substituted 4- to 8-membered non-aromatic heterocyclyl group, an unsubstituted or substituted C₃₋₈ cycloalkyl group, an unsubstituted or substituted straight or branched C₁₋₁₀alkyl, an unsubstituted or substituted C₅₋₇ cycloalkenyl or R⁵;
R⁴ is selected from hydrogen, C₁₋₆alkyl, C₃₋₆ cycloalkyl, or halosubstitutedC₁₋₆alkyl, COCH₃, or SO₂Me;
R⁵ is 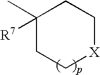
wherein p is 0, 1 or 2, and X is CH₂, O, S, SO or SO₂;
R⁶ is (C₃₋₆)cycloalkyl, 4- to 7-membered non aromatic heterocyclic group or unsubstituted C₂₋₆alkyl or substituted (C₁₋₆)alkyl;
R⁷ is OH, C₁₋₆alkoxy, NR^{8a}R^{8b}, NHCOR⁹, NHSO₂R⁹, SOqR⁹;
R^{8a} is H or C₁₋₆alkyl;
R^{8b} is H or C₁₋₆alkyl;
R⁹ is C₁₋₆alkyl;
q is 0, 1 or 2;
and pharmaceutically acceptable derivatives thereof,
wherein R⁶ is not CHxFn wherein n is 1, 2, or 3, x is 0, 1 or 2 and n and x add up to 3.

2. A compound as claimed in claim 1 wherein X is CH₂, O or S.

3. A compound as claimed in claim 1 wherein R⁶ is (C₃₋₆)cycloalkyl, 4- to 7-membered non aromatic heterocyclic group or unsubstituted C₂₋₆alkyl.

4. A compound as claimed in claim 1 wherein compounds of formula (I) are compounds of formula (Ia): 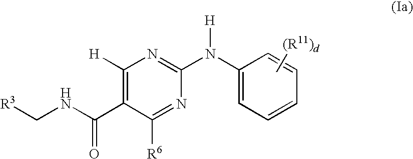
wherein:
R³ is an unsubstituted or substituted 4- to 8-membered non-aromatic heterocyclyl group;
R⁶ is unsubstituted or substituted C₂₋₆alkyl;
R¹¹ is selected from halo, cyano, methyl, trifluoromethyl, methoxy or trifluoromethoxy;
d is 0, 1, 2 or 3;
and pharmaceutically acceptable derivatives thereof.

5. A compound as claimed in claims 1 wherein R⁶ is either ethyl, substituted ethyl, isopropyl or tert-butyl.

6. (canceled)

7. A pharmaceutical composition comprising a compound as claimed in claim 1 or a pharmaceutically acceptable derivative thereof and a pharmaceutical carrier or diluent thereof.

8. A pharmaceutical composition as claimed in claim 7 further comprising a second therapeutic agent.

9. A method of treating a human or animal subject suffering from a condition which is mediated by the activity of cannabinoid 2 receptors which comprises administering to said subject a therapeutically effective amount of a compound of formula (I) as claimed in claim 1 or a pharmaceutically acceptable derivative thereof.

10. A method of treating a human or animal subject suffering from an immune disorder, an inflammatory disorder, pain, rheumatoid arthritis, multiple sclerosis, osteoarthritis or osteoporosis which method comprises administering to said subject an effective amount of a compound of formula (I) as claimed in claim 1 or a pharmaceutically acceptable derivative thereof.

11. A pharmaceutical composition comprising a compound as claimed in claim 4 or a pharmaceutically acceptable derivative thereof and a pharmaceutical carrier or diluent thereof.

12. A pharmaceutical composition as claimed in claim 11 further comprising a second therapeutic agent.

13. A method of treating a human or animal subject suffering from a condition which is mediated by the activity of cannabinoid 2 receptors which comprises administering to said subject a therapeutically effective amount of a compound of formula (Ia) as claimed in claim 4 or a pharmaceutically acceptable derivative thereof.

14. A method of treating a human or animal subject suffering from an immune disorder, an inflammatory disorder, pain, rheumatoid arthritis, multiple sclerosis, osteoarthritis or osteoporosis which method comprises administering to said subject an effective amount of a compound of formula (Ia) as claimed in claim 4 or a pharmaceutically acceptable derivative thereof.

15. A pharmaceutical composition comprising a compound as claimed in claim 1 or a pharmaceutical derivative thereof and at least one Cox-2 inhibitor.

16. A pharmaceutical composition comprising a compound as claimed in claim 1 or a pharmaceutical derivative thereof and at least one PDE4 inhibitor.

17. A pharmaceutical composition comprising a compound as claimed in claim 4 or a pharmaceutical derivative thereof and at least one Cox-2 inhibitor.

18. A pharmaceutical composition comprising a compound as claimed in claim 4 or a pharmaceutical derivative thereof and at least one PDE4 inhibitor.
